(19) United States
(12) Patent Application Publication
(10) Pub. No.: US 2025/0295417 A1
(43) Pub. Date: Sep. 25, 2025

(54) SURGICAL CLIP CARTRIDGE AND METHOD OF USE

(71) Applicant: TELEFLEX MEDICAL INCORPORATED, Morrisville, NC (US)

(72) Inventors: Adam R. Dunki-Jacobs, Cincinnati, OH (US); Matthew Edward Griffey, Waco, TX (US)

(21) Appl. No.: 19/083,150

(22) Filed: Mar. 18, 2025

Related U.S. Application Data
(60) Provisional application No. 63/567,313, filed on Mar. 19, 2024.

(51) Int. Cl. A61B 17/128

(52) U.S. Cl. CPC A61B 17/1285

(57) ABSTRACT

The present disclosure relates to a method, system and surgical clip apparatus for loading surgical clips in a clip applier intracorporeally, the surgical clip apparatus including a cartridge body having a distal end, a proximal end, and a cavity for storing surgical clips inside; a distal cap attached to the cartridge body, the distal cap providing a retaining force to the surgical clip when the distal cap is in a first state in which the clip applier does not interact with the distal cap, and the distal cap reducing the retaining force to the surgical clip when the distal cap is in a second state in which the clip applier interacts with the distal cap during loading of the clip to the clip applier; and a clip advancing mechanism for advancing the clip within the cavity of the cartridge body toward the distal end of the cartridge body.

### CROSS-REFERENCE TO RELATED APPLICATION

This application claims the benefit of U.S. Provisional Patent Application No. 63/567,313 filed on Mar. 19, 2024, the disclosure of which is hereby incorporated by reference in its entirety.

### TECHNICAL FIELD

The present disclosure generally relates to an apparatus for storing and dispensing surgical clips, and more particularly, to a surgical clip cartridge for intracorporeally loading a surgical clip applier with a surgical clip.

### BACKGROUND

Many surgical procedures require vessels or other tissues of the human body to be ligated during the surgical process. For example, many surgical procedures require cutting blood vessels (e.g., veins or arteries), and these blood vessels may require ligation to stop or reduce bleeding. In some instances, a surgeon may wish to ligate the vessel temporarily to reduce blood flow to the surgical site during the surgical procedure. In other instances, a surgeon may wish to permanently ligate a vessel. Ligation of vessels or other tissues can be performed by closing the vessel with a surgical clip, or by suturing the vessel with surgical thread. The use of surgical thread for ligation requires complex manipulations of the needle and suture material to form the knots required to secure the vessel. Such complex manipulations are time-consuming and difficult to perform, particularly in endoscopic surgical procedures, which are characterized by limited space and visibility. By contrast, surgical ligation clips are relatively easy and quick to apply. Accordingly, the use of ligation clips in endoscopic as well as open surgical procedures has grown dramatically.

A surgical clip cartridge is a type of holding device that is typically used to store and retain multiple clips between the time of their manufacture and/or packaging and ultimate use in a surgical procedure. Some conventional surgical clip cartridges are intended for use with manual clip appliers that receive one clip at a time between the jaws, and which have to be reloaded manually after the previous clip has been crimped. These manual clip appliers usually have a forceps-type design and the reloading operation is generally accomplished by inserting the jaws of the applier into the clip cartridge and engaging or grasping a clip contained therein. The jaws of the clip applier generally have longitudinal grooves to receive the clip legs and may have end-dams at the distal end of each groove to limit distal movement of the clip. The clip is secured in the jaws by the natural resiliency of the clip legs and by the end-dams if they are present. The jaws of the clip applier may also include recesses for engaging or gripping corresponding bosses on the clip legs.

Conventional clip cartridges are typically handheld by an operator or placed on a surface, such as a table or a tray, in the operating room while the surgical clips are manually loaded onto a clip applier. Such conventional clip cartridges are prone to being dropped, misplaced, or contaminated during a surgical procedure. Furthermore, such conventional clip cartridges are intended to supply clips to a clip applier extracorporeally. Stated another way, in a manual ligation procedure, conventional clip cartridges are maintained outside the patient and away from the surgical site. The surgeon is therefore restricted to loading a clip from the clip cartridge onto the clip applier outside the patient. Once the clip has been manually loaded from the cartridge onto the clip applier, the clip applier together with the clip is inserted into the patient to perform a ligation. However, this procedure requires the surgeon to divert attention back and forth from the surgical site to the clip cartridge outside of the patient for reloading, resulting in time loss and direct visualization loss.

Some surgical procedures are carried out within an intracorporeal space of the patient. For instance, a trocar may provide access for a clip applier to enter into a body cavity of the patient. Such intracorporeal procedures are sometimes carried out using a robotic platform employing a polymer clip applier, wherein the jaws of clip applier are able to access the intracorporeal space by inserting the clip applier through the trocar. However, conventional robotic polymer clip appliers need to be loaded manually outside of the body cavity. In other words, surgeons or operating room staff need to manually reload the clip applier by retracting the end effector of the clip applier outside the patient to reload the device. This process requires removing the clip applier from the robot arm and reloading surgical clips manually to the clip applier. This process is time consuming and interrupts the flow of the surgical procedure, which can be dangerous for the patient. In many cases, surgeons have assistants who use laparoscopic clip appliers instead of the robotic appliers for this reason. However, it is preferential for the surgeon to control the application of clips to the clip applier rather than having their assistants do it. Indeed, some surgeons even prefer first assistants to fire the clip applier manually because of this inefficiency. There are currently no known multi-fire clip appliers for robotic platforms due to these aforementioned restrictions.

The present invention solves these problems by providing a ligation clip cartridge that is introduced through a trocar to enable loading of a robotic clip applier intracorporeally, thus eliminating the need to remove the clip applier from the body cavity of the patient to reload. As such, the present invention contemplates an improved surgical clip cartridge that allows quicker clip loading with no visualization loss. The clip cartridge of the present invention also improves the ability of the surgeon to use an existing robotic polymer clip applier without impacting the flow of the surgical procedure by giving them full control of the clip loading process.

### SUMMARY

The foregoing needs are met, to a great extent, by the present disclosure of a ligation clip cartridge discussed herein. The present invention is generally directed to a surgical clip apparatus, method, and system for loading at least one surgical clip into a clip applier. According to one aspect, a surgical apparatus for loading a surgical clip to a clip applier comprises a cartridge body including a distal end, a proximal end, and a cavity configured to store the surgical clip inside the cartridge body; a distal cap attached to the cartridge body, the distal cap configured to provide a retaining force to the surgical clip when the distal cap is in a first state in which the clip applier does not interact with the distal cap, and the distal cap configured to reduce the retaining force to the surgical clip when the distal cap is in a second state in which the clip applier interacts with the distal cap during loading of the clip to the clip applier; and a clip advancing mechanism configured to advance the clip within the cavity of the cartridge body toward the distal end of the cartridge body.

According to another aspect, the distal cap includes a flexible retaining arm configured to provide the retaining force to a side of the surgical clip, and a clip retainer cap configured to reduce the retaining force to the side of the surgical clip.

According to another aspect, the clip retainer cap comprises at least one guiding surface configured to interact with an end effector of the clip applier to deflect the clip retainer cap in a lateral direction away from the surgical clip.

According to another aspect, the distal cap further comprises an anchor cap fixed to the cartridge body.

According to another aspect, the flexible retaining arm extends between the anchor cap and the clip retainer cap.

According to another aspect, the distal cap further comprises at least one inner surface configured to engage at least one boss of the surgical clip when the distal cap is in the first state and not engage the at least one boss of the surgical clip when the distal cap is in the second state.

According to another aspect, the clip advancing mechanism comprises a loading spring configured to provide a loading force to the surgical clip; a rod extending from the proximal end of the cartridge body; and a clip advancer attached to a distal end of the rod and configured to transmit the loading force to the surgical clip and load the surgical clip to the clip applier when the distal cap is in the second state.

According to another aspect, the retaining force is greater than the loading force when the distal cap is in the first state.

According to another aspect, the retaining force is less than the loading force when the distal cap is in the second state.

According to another aspect, the clip advancer moves the surgical clip toward the distal end of the cartridge body when the distal cap is in the second state.

According to another aspect, the cavity of the cartridge body is configured to store a plurality of surgical clips, and wherein the distal cap is configured to load a distal-most surgical clip of the plurality of surgical clips to the clip applier when the distal cap is in the second state.

According to another aspect, a proximal cap is attached to the proximal end of the cartridge body.

According to another aspect, the proximal cap is integral with the cartridge body.

According to another aspect, the proximal cap is a separate part from the cartridge body.

According to another aspect, the proximal cap has a holding surface configured to be held by a grasper, a bulldog clamp, or a suture.

According to another aspect, the proximal cap is configured to be attached to a shaft.

According to another aspect, the proximal cap is attached to the shaft by an articulation joint with one or more degrees of freedom.

According to another aspect, the cartridge body is cylindrical.

According to another aspect, the cartridge body comprises at least one rib to assist being held in place by graspers.

According to another aspect, a method of loading a surgical clip to a clip applier intracorporeally comprises inserting the clip applier through a first trocar into an intracorporeal space; inserting the surgical apparatus of claim 1 through a second trocar into the intracorporeal space; orienting end effectors of the clip applier in an open position proximal to the retainer cap of the surgical apparatus; actuating the end effectors toward a closed position so that the end effectors interact with at least one guiding surface of the retainer cap to allow the surgical apparatus to enter the second state; receiving the surgical clip into the end effectors; and releasing the end effectors to allow the surgical clip apparatus to enter the first state.

According to another aspect of the method, the surgical clip is manually inserted into the cavity prior to the surgical apparatus being inserted into the intracorporeal space.

According to another aspect, a system for performing a medical procedure intracorporeally comprises a surgical clip cartridge apparatus; a clip applier; and an insertion mechanism configured to store the at least one surgical clip in an uncompressed state prior to the medical procedure and insert the at least one surgical clip into the surgical clip cartridge apparatus intracorporeally.

According to another aspect, the insertion mechanism comprises an interface including at least one ramp configured to deflect the at least one guiding surface of the clip retainer cap in a lateral direction and hold the surgical apparatus in the second state.

According to another aspect, the insertion mechanism further comprises a track configured to guide the surgical clip during storage and during insertion of the surgical clip into the surgical apparatus.

According to another aspect, the insertion mechanism further comprises a clip inserter configured to move the surgical clip from the insertion mechanism into the cavity of the surgical apparatus in a partially compressed state.

### BRIEF DESCRIPTION OF THE DRAWINGS

In order that the present disclosure may be readily understood, aspects of the ligation clip cartridge are illustrated by way of examples in the accompanying drawings.

FIG. 1 illustrates a cross-sectional view along the longitudinal axis of the surgical clip apparatus.

FIG. 2 illustrates a cross-sectional view of the proximal cap and proximal end of the cartridge body including the clip advancing mechanism.

FIG. 3 illustrates a cross-sectional view of the cartridge body including the clip advancing mechanism.

FIG. 4 illustrates a cross-sectional view of the distal cap of the surgical clip apparatus.

FIG. 5 illustrates a cross-sectional view across A-A in FIG. 4 of the distal cap of the surgical apparatus.

FIG. 6 illustrates a perspective view of the surgical clip apparatus.

FIG. 7 illustrates a perspective view of the distal cap of the surgical clip apparatus.

FIG. 8 illustrates a perspective view of the distal cap of the surgical clip apparatus and the end effectors of a clip applier.

FIG. 9A illustrates a front view of the distal cap of the surgical clip apparatus in the deactivated state and the end effectors of a clip applier in the open position.

FIG. 9B illustrates a front view of the distal cap of the surgical clip apparatus in the activated state and the end effectors of a clip applier in the closed position.

FIG. 10 illustrates a perspective view of the surgical clip apparatus in an anchored position.

FIG. 11 illustrates a perspective view of a holding device of the surgical clip apparatus.

FIG. 12 illustrates a surgical area including trocars used to perform the surgical procedure with the surgical clip apparatus.

FIG. 13 illustrates a method of loading surgical clips from the surgical clip apparatus to the clip applier.

FIG. 14 illustrates a perspective view of a system including the surgical clip apparatus and insertion mechanism in a released position for loading surgical clips into the surgical clip apparatus.

FIG. 15 illustrates perspective view of a system including the surgical clip apparatus and insertion mechanism in a locked position for loading surgical clips into the surgical clip apparatus.

FIG. 16 illustrates a perspective view of a clip compression slide of the insertion mechanism in a released position for loading surgical clips into the surgical clip apparatus.

FIG. 17 illustrates a perspective view of the insertion mechanism for loading surgical clips into the surgical clip apparatus.

FIG. 18 illustrates another perspective view of the insertion mechanism for loading surgical clips into the surgical clip apparatus.

### DETAILED DESCRIPTION

The present disclosure will now be described with reference to the figures, in which like reference numerals may refer to like parts throughout. The present invention is generally directed to a surgical apparatus 100, method, and system for loading a surgical clip 200 to a clip applier 300. More particularly, the surgical apparatus may be a surgical clip cartridge 100 which is configured for use in loading a surgical clip 200 to a robotic clip applier 300 within the intracorporeal space 600 during a surgical procedure. The surgical clip cartridge 100 thus improves the ability of the surgeon to use a robotic polymer clip applier 300 without impacting the flow of the surgical procedure by giving the surgeon full control of the clip loading process without the need to remove the clip applier 300 from outside the patient's body in order to load a clip 200 thereto.

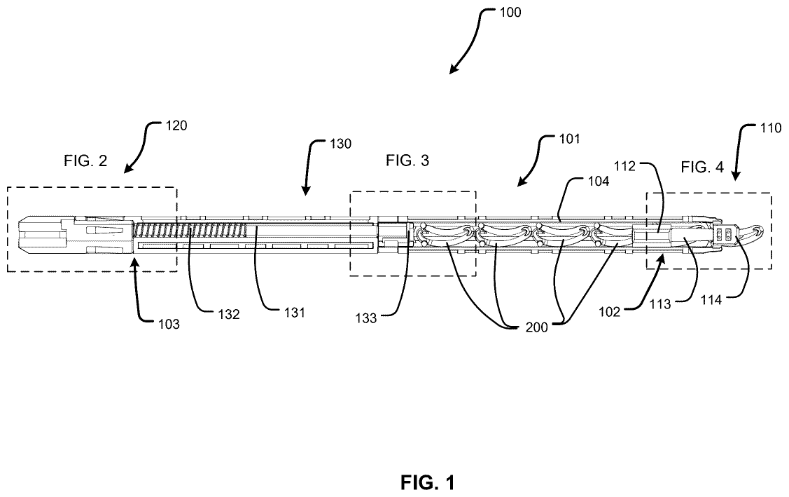

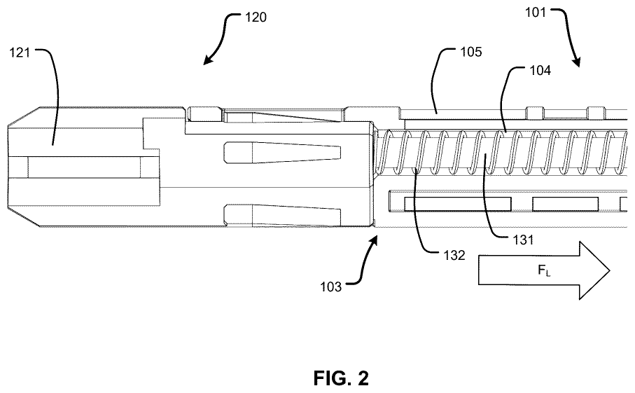

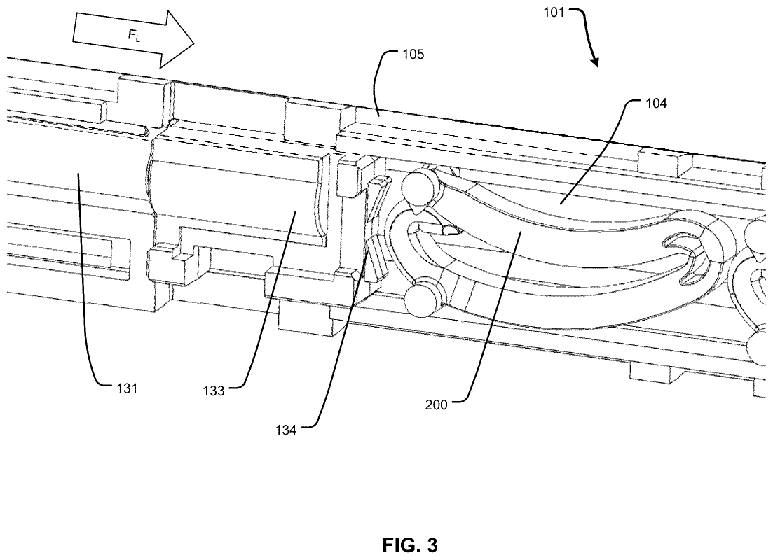

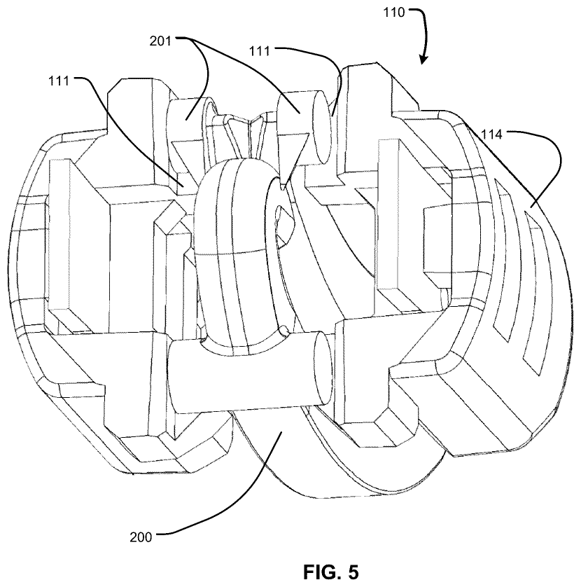

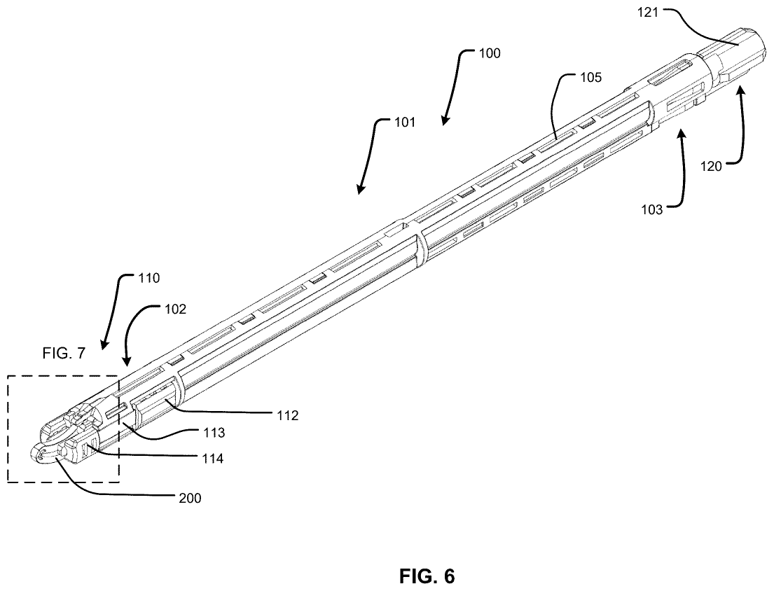

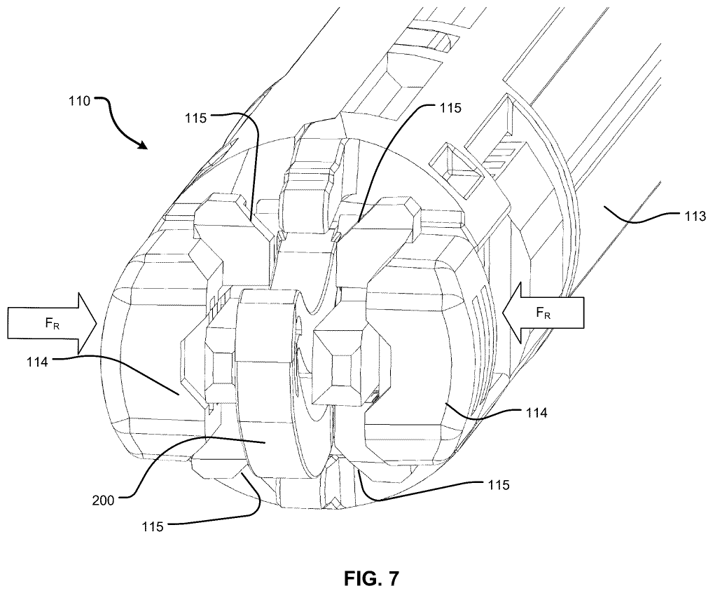

For example, as shown in FIGS. 1-7, the surgical clip cartridge 100 may include a cartridge body 101 having a distal end 102, a proximal end 103, and a cavity 104 configured to store at least one surgical clip 200, which may be manually inserted into the cavity 104 prior to the surgical clip cartridge 100 being inserted into an intracorporeal space 600. The cavity 104 may be sized to store a plurality of surgical clips 200 as may be needed by the clinician during the surgical procedure. The cartridge body 101 may be cylindrical, but may be of any other shape, such as rectangular, or other polygonal shape suitable for storing surgical clips 200. The cartridge body 101 may also include at least one grasping surface 105 such as a rib or round boss configured to allow the surgical clip cartridge 100 to be held in place. Reliable features to grasp onto the cartridge body 101 are helpful to enable the clip applier 300 or other robotic grasping tool to hold onto the surgical clip cartridge 100 during the surgical procedure and aid in loading on the surgical clips 200.

The surgical clips 200 used may be made of any suitable size and may be applied to any number of tissues, such as blood vessels, lymph nodes, nerves, cystic ducts, and cardiac tissue. The surgical clips 200 may be constructed from any suitable biocompatible material, such as metals and polymers. Moreover, the cartridge body 101 may be constructed from any suitable biocompatible material, such as metals and polymers. However, the present invention is particularly suitable for practice with polymeric clips 200.

Referring to FIGS. 1-3, at the proximal end 103 of the cartridge body 101, a proximal cap 120 may be attached or fixed to the proximal end 103, which includes a clip advancing mechanism 130. This clip advancing mechanism 130 may include a rod 131 attached to and extend distally from the proximal cap 120, a loading spring 132 configured to provide a loading force F_{L} to the surgical clip(s) 200, and a clip advancer 133 attached to a distal end 102 of the rod 131. This rod 131 helps direct the loading force F_{L} of the loading spring 132 along the longitudinal axis of the cartridge body 101 and provides additional stability to the clip advancer 133.

The loading spring 132 preferably is a helical or spiral spring, but any spring known to those of ordinary skill in the art capable of producing a linear force may be used. As such, this loading force F_{L} is biased in the distal direction of the surgical clip cartridge 100. This clip advancer 133 may further be configured to transmit the loading force F_{L} to the surgical clip(s) 200 and load the surgical clip(s) 200 into the clip applier 300 when the surgical clip cartridge 100 is in an activated state. The clip advancer 133 may include at least one engagement surface 134 which engages with the surgical clip(s) 200 during the transmission of the loading force F_{L} in order to urge the surgical clip(s) 200 through the cavity 104 to be loaded in the clip applier 300. This engagement surface 134 may be shaped in a complementary fashion with the surgical clip(s) 200 to aid in the loading operation. Moreover, the total length of the clip advancing mechanism 130 including the length of the loading spring 132, the rod 131, and the clip advancer 133 may take into account the number of surgical clip(s) 200 that are stored within the cavity 104 of the cartridge body 101 so that the final stored clip will be easily accessed by the clip applier 300. In some implementations, the proximal cap 120 may be integral with the cartridge body 101. In other implementations, the proximal cap 120 may be a separate part from the cartridge body 101.

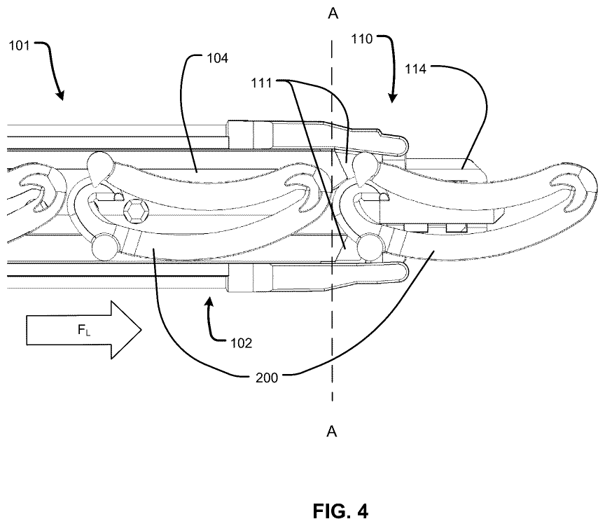

As seen in FIGS. 4-7, at the distal end 102 of the cartridge body 101, a distal cap 110 may be attached to the cartridge body 101 and may include a flexible retaining arm 113 configured to provide a retaining force F_{R} on the surgical clip(s) 200 inside the cartridge body 101 when the surgical clip cartridge 100 is in a first or deactivated state. According to some aspects, this flexible retaining arm 113 may include at least one leaf spring, but any spring or other flexible device capable of providing a lateral force may be used. The flexible retaining arm 113 is also biased toward the center of the surgical clip cartridge 100 to provide the retaining force F_{R} necessary to confine the surgical clip(s) 200 inside the cartridge body 101 when the surgical clip cartridge 100 is in the deactivated state.

Furthermore, the distal cap 110 may also include a clip retainer cap 114 configured to reduce the retaining force F_{R} on the surgical clip(s) 200 when the surgical clip cartridge 100 is in a second or activated state. The distal cap 110 may further include an anchor cap 112 fixed to the cartridge body 101, where the flexible retaining arm 113 may be configured to extend between the anchor cap 112 and the clip retainer cap 114. In particular, the flexible retaining arm 113 may be a plurality of leaf springs or the like, which provide opposing lateral forces toward the center of the surgical clip cartridge 100. The clip retainer cap 114 may include at least one guiding surface 115 which may interact with the end effectors or jaws 301 of the clip applier 300 to actuate the surgical clip cartridge 100 between the deactivated state and the activated state. The at least one guiding surface 115 may be any shape or configuration which complements the jaws or end effectors 301 of a clip applier 300, such as curved, chamfered, beveled edges, or others known to a person of ordinary skill in the art.

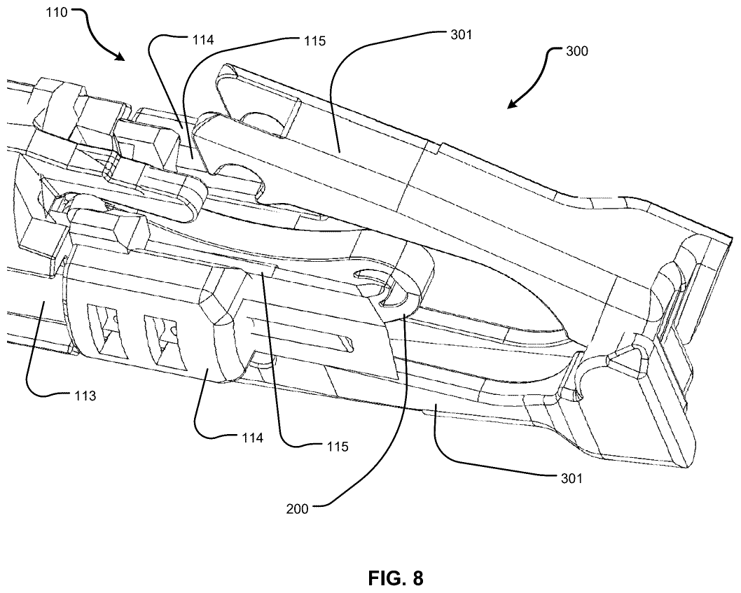

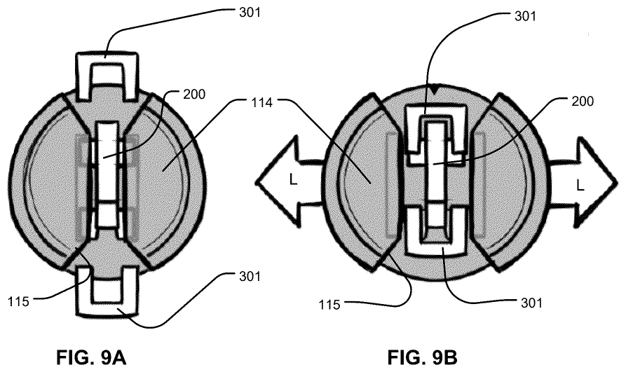

For example, as seen in FIGS. 8-9B, the flexible retaining arm 113 comprises a pair of leaf springs each attached to the distal cap 110. In this configuration, the distal cap 110 has an anchor cap 112 made of two parts attached to the cartridge body 101 on opposite sides and a clip retainer cap 114 made of two parts, which are symmetrical along the axis of the cartridge body 101. Here, the clip retainer cap 114 includes four guiding surfaces 115, two guiding surfaces 115 on the left part of the clip retainer cap 114 and two guiding surfaces 115 on the right part of the clip retainer cap 114. Moreover, two of the guiding surfaces 115 interact with the top end effector or jaw of the clip applier 300 and the other two of the guiding surfaces 115 interact with the bottom end effector or jaw of the clip applier 300. The guiding surfaces 115 are also angled inward toward the center of the surgical clip cartridge 100 to guide the jaws or end effectors 301 of the clip applier 300 and aid in creating a lateral force opposing the retaining force F_{R} of the flexible retaining arm 113.

In this way, the surgical clip cartridge 100 enters the activated state (FIG. 9B) when the end effectors 301 close, counteract the retaining force F_{R} of the flexible retaining arm 113, and deflect the at least one guiding surface 115 in a lateral direction L and the surgical clip cartridge 100 enters back into the deactivated state (FIG. 9A) when the end effectors 301 are opened and restoring the retaining force F_{R} to the retaining spring's 113 biased direction. In aspects where a plurality of surgical clips 200 are utilized, the closest of the plurality of surgical clips 200 to the distal cap 110 may be loaded into the clip applier 300 when the end effectors 301 of the clip applier 300 close onto the at least one guiding surface 115 of the clip retainer cap 114, still in a partially compressed position. Once the end effectors 301 of the clip applier 300 are open again, this surgical clip 200 will be loaded in the clip applier 300 in its fully open position, ready for ligation. At this point, the surgical clip cartridge 100 enters back into the deactivated state, which causes the clip advancer 133 to move the next of the plurality of surgical clips 200 to move toward the distal end 102 of the cartridge body 101 to be ready to be loaded into the clip applier 300 during the next actuation of the surgical clip cartridge 100 between the activated state and deactivated state.

As such, the distal cap 110 may further include at least one inner surface 111 configured to engage at least one boss 201 of the surgical clip(s) 200 when the surgical clip cartridge 100 is in the deactivated state and not engage the at least one boss 201 of the surgical clip(s) 200 when the surgical clip cartridge 100 is in the activated state. These inner surfaces 111 of the distal cap 110 help confine and retain the surgical clip(s) 200 inside the surgical clip cartridge 100 by providing a stop until the clinician is ready to load them into the clip applier 300 and actuate the surgical clip cartridge 100. The bosses 201 of the surgical clip(s) 200 interacting with the inner surfaces 111 are located at the open end of the surgical clip 200(s). As such, the hinge portion of the surgical clip(s) 200 is oriented distally with respect to the cartridge body 101 and, when ready to be loaded in the clip applier 300, extend beyond the clip retainer cap 114 in order to be easily received by the jaws or end effectors 301 of the clip applier 300 during actuation of the surgical clip cartridge 100.

Furthermore, the flexible retaining arm 113 and the loading spring 132 are designed in a way that allows the retaining force F_{R} to be greater than the loading force F_{L} when the surgical clip cartridge 100 is in the deactivated state and the retaining force F_{R} may be less than the loading force F_{L} when the surgical clip cartridge 100 is in the activated state. This configuration allows the surgical clip(s) 200 to easily eject from the surgical clip cartridge 100 into the clip applier 300 when activated and be retained within the surgical clip cartridge 100 when it is deactivated. Such retaining force F_{R} and spring force are related to the individual properties of the springs utilized for the system and surgical clip cartridge 100.

As such, the flexible retaining arm 113 needs to have a retaining force F_{R} strong enough to quickly rebound to prevent the next clip 200 from ejecting prior to engagement with the distal cap 110 and also needs to deflect laterally with relative ease when closing the clip applier 300 on the cartridge body 101 to retrieve a clip 200. Likewise, the loading spring 132 needs to be strong enough to urge the surgical clip(s) 200 forward, but weak enough to not overcome the flexible retaining arm 113 when the surgical clip cartridge 100 is in the deactivated state. Thus, the surgical clip cartridge 100 reliably ejects the surgical clip(s) 200 to load in the clip applier 300 with ease and helps provide the clinician with a visual and tactile indication of the loading process. Moreover, the retaining force F_{R} can be increased by increasing the thickness or width of the flexible retaining arm 113 or reducing the effective moment arm of the flexible retaining arm 113; and vice versa. Similarly, the loading force F_{L} can be increased by using a thicker diameter loading spring 132, reducing the number of active coils of the loading spring 132, decreasing the diameter of the active coils of the loading spring 132; and vice versa. The retaining force F_{R} and loading force F_{L} may also be changed by the material of either spring, where the higher the modulus of elasticity will increase the force utilized.

Another clinical concern in loading surgical clips intracorporeally is the potential for accidentally leaving the clip cartridge inside the intracorporeal space, such as a patient's abdomen, during the surgery. However, the introduction of the clip cartridge of the present invention into the intracorporeal space by using a trocar avoids this problem. In some implementations, for instance, the clip cartridge may be affixed to tissue using a suture, clamp, or hook, among others to help prevent losing the cartridge during portions of the surgery where the cartridge is not utilized. Furthermore, a suture may serve as a tether to remove the clip cartridge after use.

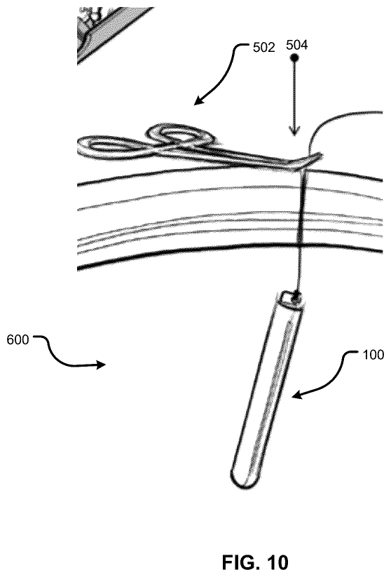

In particular, the proximal cap 120 may have a holding surface 121 configured to be held in an anchored position by a grasper 502, bulldog clamp 503, or a suture 504 (FIG. 10), or may be configured to be attached to a shaft 501. These methods help keep track 403 of the surgical clip cartridge 100 during the surgery when the surgical clip cartridge 100 is not in use to avoid the surgical clip cartridge 100 from being lost inside the intracorporeal space 600. For example, in the case of the use of a suture 504, in FIG. 10, a suture 504 is tied to the holding surface 121, which is fed from the surgical clip cartridge 100 in the intracorporeal space 600, such as an abdominal cavity, to the outside of the body 101. Such a suture 504 may be attached to the holding surface 121 well prior to use or attached by the clinician immediately before use of the surgical clip cartridge 100. A grasper 502, bulldog clamp 503, or other device may be used to clamp the suture 504 to adjust the appropriate length of the suture 504 and keep the surgical clip cartridge 100 out of the surgical area when not in use.

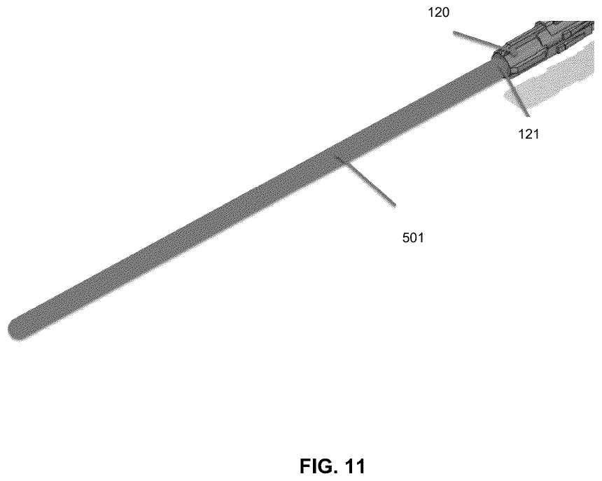

In another manner, a clamp, such as a bulldog clamp 503 may be physically attached to the holding surface 121, which enables the clinician to find an area within the intracorporeal space 600 outside the surgical area to position the surgical clip cartridge 100 when not in use. For example, as seen in FIG. 11, the proximal cap 120 may be attached to the shaft 501 by an articulation joint with one or more degrees of freedom.

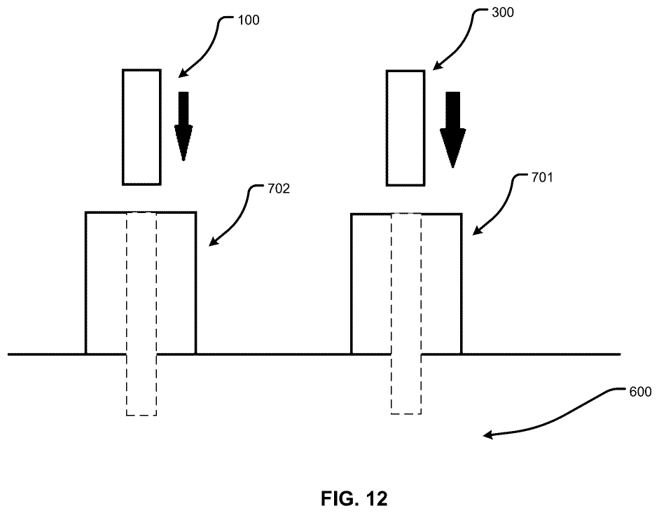

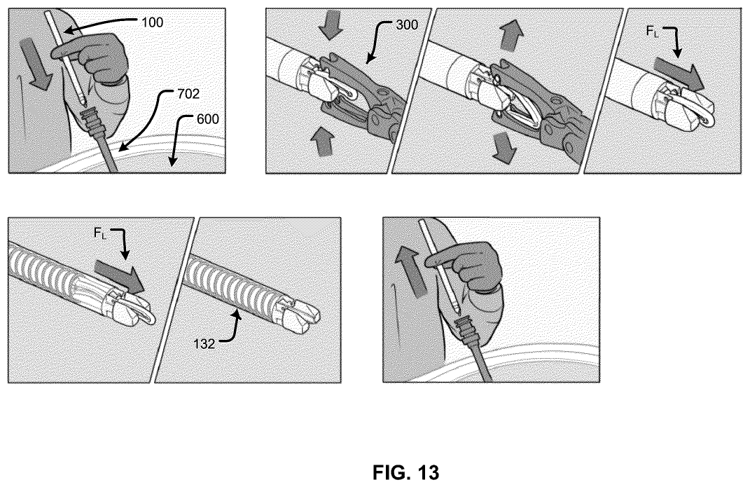

Another aspect of the invention relates to a method of loading surgical clip(s) 200 into a clip applier 300 intracorporeally. As seen in FIG. 12, the first step involves preparing the surgical area for the procedure in creating a sterile field. Within this area, two trocars 701, 702, such as laparoscopic trocars, one for inserting clip applier 300, such as those used during robotic surgery, and one for inserting the surgical clip cartridge 100. The rest of the method may be seen in FIG. 13, after inserting the clip applier 300 through the first trocar 701 into an intracorporeal space 600. For example, the method includes inserting the surgical clip cartridge 100 through the second trocar 702 into the intracorporeal space 600, orienting the end effectors 301 of the clip applier 300 in an open position proximal to the clip retainer cap 114 of the distal cap 110 of the surgical clip cartridge 100, actuating the end effectors 301 toward a closed position so that the end effectors 301 interact with at least one guiding surface 115 of the clip retainer cap 114 to allow the surgical clip cartridge 100 to enter the activated state, receiving the surgical clip 200 into the end effectors 301 in a partially compressed state, and releasing the end effectors 301 to allow the surgical clip cartridge 100 to enter the deactivated state and allowing the surgical clip 200 to return to a fully decompressed state within the clip applier 300. The method may be repeated as many times as necessary depending on how many surgical clips 200 are desired to be used by the clinician during the surgical procedure. In such a case, the next clip 200 stored within the surgical clip cartridge 100 will move distally to be received by the clip applier 300 during the next actuation sequence until the cartridge is empty. After the surgical clip cartridge 100 is used, the clinician may remove it from the intracorporeal space 600 through the second trocar 702.

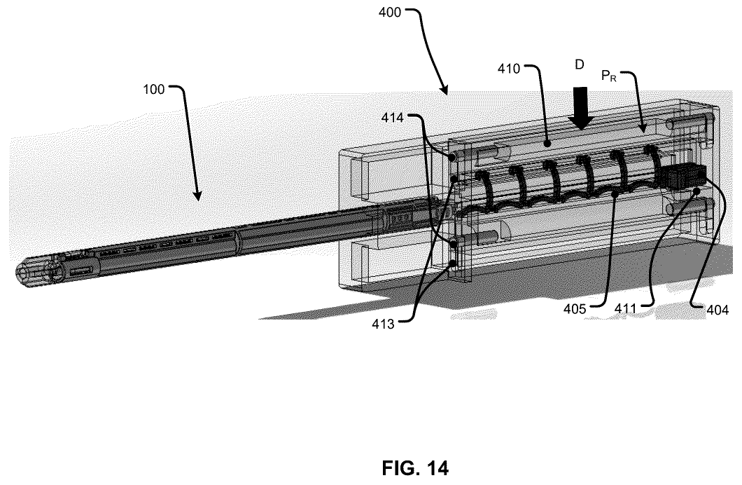

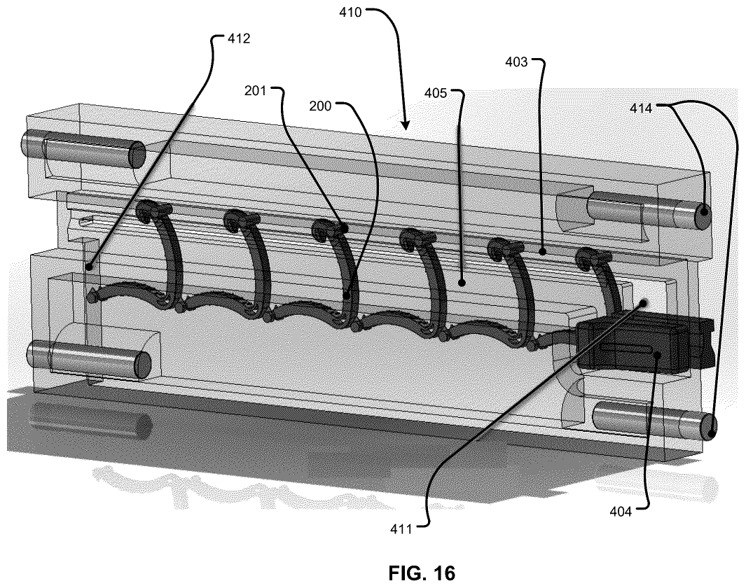

Another aspect of the invention, seen in FIGS. 14-17 relates to a system for performing a medical procedure intracorporeally including the surgical clip cartridge 100 described herein, a clip applier 300, and a clip insertion mechanism 400 configured to store the at least one surgical clip 200 in an uncompressed state prior to the medical procedure. The clip insertion mechanism 400 is configured to load the at least one surgical clip 200 into the surgical clip cartridge 100 outside the patient (i.e., in the sterile field of the surgical procedure, such as on a back table). Storing the surgical clip(s) 200 in an uncompressed state is preferable because it prevents the surgical clip(s) 200 from taking a set and not open correctly to the original boss 201-to-boss 201 distance. Thus, this insertion mechanism 400 helps load the cartridge body 101 with surgical clip(s) 200 only when needed at the time immediately prior to use.

This insertion mechanism 400 works by storing the clips 200 in an uncompressed state prior to use. At time of use, the surgical clip cartridge 100 can be inserted into the insertion mechanism 400 through a snap fit, so the surgical clip cartridge 100 remains properly installed during the insertion and storage of the surgical clip(s) 200 into the surgical clip cartridge 100.

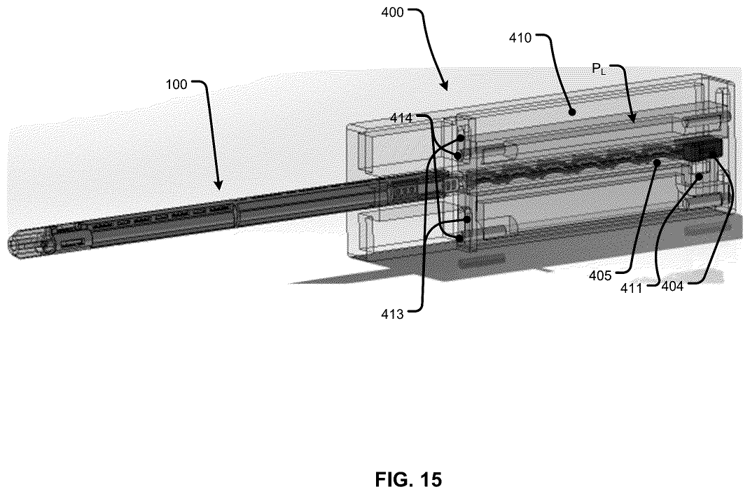

As seen in FIGS. 14-18, a clip compression slide 410 may be provided which compresses the surgical clip(s) 200 so that they can be installed in the cartridge body 101 of the surgical clip cartridge 100. This slide 410 also may include a clip trap door 411 and clip inserter lockout 412. During use of the insertion mechanism 400, the user can compress the clip 200(s) from a fully open position (FIG. 14) to a partially closed position for installing into the cartridge body 101 (FIG. 15) by sliding the clip compression slide 410 in a downward direction D from a released position P_{R} to a locked position P_{L}. The clip compression slide 410 will lock by means of a mechanical snap, detent, or some other mechanism.

To aid in the movement between the released position P_{R} and the locked position P_{L}, the clip compression slide 410 may also be provided with at least one clip compression track 413 and at least one clip compression guide 414. For example, in FIGS. 14-17, the clip compression track 413 may include two pairs of tracks on opposite sides of the clip track 403 and may include two pairs of clip compression guides 414 that slide through their respective compression tracks 413 when the clip compression slide 410 is moved between the released position P_{R} and the locked position P_{L}. The clip compression tracks 413 and the clip compression guides 414 may be of complementary shapes. For example, in FIGS. 14-17, the clip compression guides 414 may be cylindrical pegs, the clip compression tracks 413 may be elongate slots with curved ends.

Until the clip compression slide 410 is moved fully to compress the surgical clip(s) 200, the cutout in the clip compression slide 410 forms a lockout 412 for the clip inserter 404 to prevent the clip inserter 404 from moving. Similarly, the clip trap door 411 may include a surface to engage with the surgical clip(s) 200 to prevent them from moving until the clip compression slide 410 is fully compressed. Finally, once the clip compression slide 410 is moved fully to the locked position P_{L}, the clip inserter slot 405 allows for the clip inserter 404 to push clips 200 into the cartridge body 101 of the surgical clip cartridge 100 and releasing the clips 200 from the clip trap door 411 and the clip inserter 404 from the lockout 412.

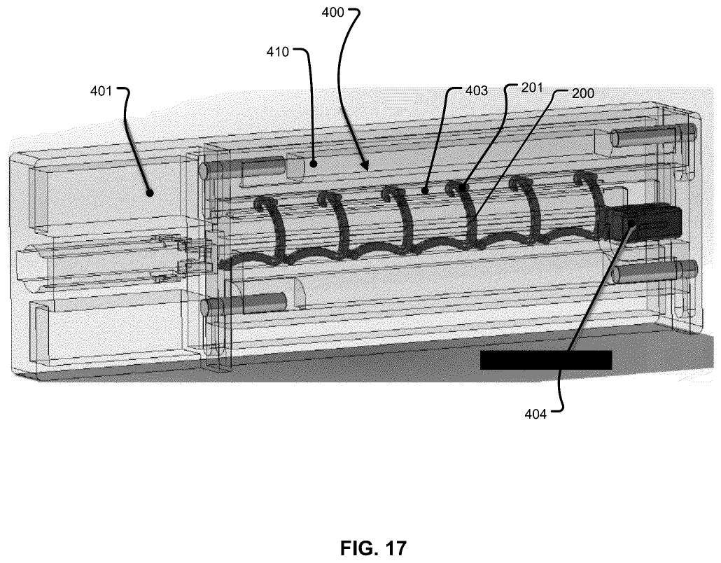

Moreover, as best seen in FIG. 17, the insertion mechanism 400 may also include a track 403 configured to guide the at least one surgical clip 200 during storage and during insertion of the surgical clip(s) 200 into the surgical clip cartridge 100 by engaging with the bosses 201 of the surgical clip(s) 200 to ensure the surgical clip(s) 200 do not move freely during transport, storage, and handling. The insertion mechanism 400 may also include a clip inserter 404 at the end of the track 403 and the slot 405 for the clip inserter 404. This inserter 404 may configured to move the at least one surgical clip 200 from the insertion mechanism 400 into the cavity 104 of the surgical clip cartridge 100 in a partially compressed state.

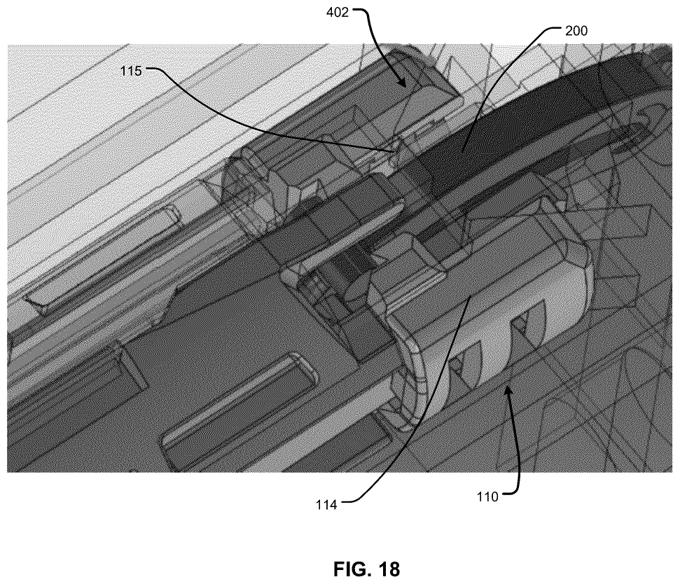

Moreover, as seen in FIG. 18 for example, the insertion mechanism 400 may include an interface 401 with at least one ramp 402 configured to deflect the at least one guiding surface 115 of the clip retainer cap 114 in a lateral direction L and hold the surgical clip cartridge 100 in the activated state. This ramp 402 feature spreads apart the distal cap 110 during insertion of the surgical clip(s) 200 into the surgical clip cartridge 100 and allows the surgical clip(s) 200 to be installed from the distal end 102 of the cartridge body 101. However, in an alternative, the ramp 402(s) may not be necessary in the case where the distal cap 110 have lead-in surfaces that deflect when surgical clip(s) 200 are push inserted from the distal end 102 of the cartridge body 101. Once the surgical clip(s) 200 are fully inserted, the flexible retaining arm 113 springs the distal cap 110 back to the deactivated state of the surgical clip cartridge 100 to allow the retention and storage of the surgical clip(s) 200 via the bosses 201 of the surgical clip(s) 200 and inner surface(s) 111 of the distal cap 110.

While the surgical clip cartridge has been described in terms of what may be considered to be specific aspects, the present disclosure is not limited to the disclosed aspects. Additional modifications and improvements to the surgical clip cartridge may be apparent to those skilled in the art. Moreover, the many features and advantages of the disclosure are apparent from the detailed specification, and thus, it is intended by the appended claims to cover all such features and advantages of the disclosure which fall within the spirit and scope of the disclosure. Further, it is not desired to limit the disclosure to the exact construction and operation illustrated and described, and accordingly, all suitable modifications and equivalents may be resorted to, falling within the scope of the disclosure. Accordingly, the present disclosure should be considered as illustrative and not restrictive. As such, this disclosure is intended to cover various modifications and similar arrangements included within the spirit and scope of the claims, which should be accorded their broadest interpretation so as to encompass all such modifications and similar structures.

What is claimed is:

1. A surgical apparatus for loading a surgical clip to a clip applier, the apparatus comprising:
a cartridge body including a distal end, a proximal end, and a cavity configured to store the surgical clip inside the cartridge body;
a distal cap attached to the cartridge body, the distal cap configured to provide a retaining force to the surgical clip when the distal cap is in a first state in which the clip applier does not interact with the distal cap, and the distal cap configured to reduce the retaining force to the surgical clip when the distal cap is in a second state in which the clip applier interacts with the distal cap during loading of the clip to the clip applier; and
a clip advancing mechanism configured to advance the clip within the cavity of the cartridge body toward the distal end of the cartridge body.

2. The surgical apparatus according to claim 1, wherein the distal cap includes a flexible retaining arm configured to provide the retaining force to a side of the surgical clip, and a clip retainer cap configured to reduce the retaining force to the side of the surgical clip.

3. The surgical apparatus according to claim 2, wherein the clip retainer cap comprises at least one guiding surface configured to interact with an end effector of the clip applier to deflect the clip retainer cap in a lateral direction away from the surgical clip.

4. The surgical apparatus according to claim 1, wherein the distal cap further comprises an anchor cap fixed to the cartridge body.

5. The surgical apparatus according to claim 4, wherein the flexible retaining arm extends between the anchor cap and the clip retainer cap.

6. The surgical apparatus according to claim 1, wherein the distal cap further comprises at least one inner surface configured to engage at least one boss of the surgical clip when the distal cap is in the first state and not engage the at least one boss of the surgical clip when the distal cap is in the second state.

7. The surgical apparatus according to claim 1, wherein the clip advancing mechanism comprises:
a loading spring configured to provide a loading force to the surgical clip;
a rod extending from the proximal end of the cartridge body; and
a clip advancer attached to a distal end of the rod and configured to transmit the loading force to the surgical clip and load the surgical clip to the clip applier when the distal cap is in the second state.

8. The surgical apparatus according to claim 7, wherein the retaining force is greater than the loading force when the distal cap is in the first state.

9. The surgical apparatus according to claim 8, wherein the retaining force is less than the loading force when the distal cap is in the second state.

10. The surgical apparatus according to claim 7, wherein the clip advancer moves the surgical clip toward the distal end of the cartridge body when the distal cap is in the second state.

11. The surgical apparatus according to claim 1, wherein the cavity of the cartridge body is configured to store a plurality of surgical clips, and wherein the distal cap is configured to load a distal-most surgical clip of the plurality of surgical clips to the clip applier when the distal cap is in the second state.

12. The surgical apparatus according to claim 1, further comprising a proximal cap attached to the proximal end of the cartridge body.

13. The surgical apparatus according to claim 12, wherein the proximal cap is integral with the cartridge body.

14. The surgical apparatus according to claim 12, wherein the proximal cap is a separate part from the cartridge body.

15. The surgical apparatus according to claim 12, wherein the proximal cap has a holding surface configured to be held by a grasper, a bulldog clamp, or a suture.

16. The surgical apparatus according to claim 12, wherein the proximal cap is configured to be attached to a shaft.

17. The surgical apparatus according to claim 16, wherein the proximal cap is attached to the shaft by an articulation joint with one or more degrees of freedom.

18. The surgical apparatus according to claim 1, wherein the cartridge body is cylindrical.

19. The surgical clip apparatus according to claim 1, wherein the cartridge body comprises at least one rib to assist being held in place by graspers.

20. A method of loading a surgical clip to a clip applier intracorporeally, the method comprising:
inserting the clip applier through a first trocar into an intracorporeal space;
inserting the surgical apparatus of claim 1 through a second trocar into the intracorporeal space;
orienting end effectors of the clip applier in an open position proximal to the retainer cap of the surgical apparatus;
actuating the end effectors toward a closed position so that the end effectors interact with at least one guiding surface of the retainer cap to allow the surgical apparatus to enter the second state;
receiving the surgical clip into the end effectors; and
releasing the end effectors to allow the surgical clip apparatus to enter the first state.

21. The method according to claim 20, wherein the surgical clip is manually inserted into the cavity prior to the surgical apparatus being inserted into the intracorporeal space.

22. A system for performing a medical procedure intracorporeally, the system comprising:
the surgical apparatus of claim 1;
a clip applier; and
an insertion mechanism configured to store the at least one surgical clip in an uncompressed state prior to the medical procedure and insert the at least one surgical clip into the surgical clip apparatus intracorporeally.

23. The system according to claim 22, wherein the insertion mechanism comprises an interface including at least one ramp configured to deflect the at least one guiding surface of the clip retainer cap in a lateral direction and hold the surgical apparatus in the second state.

24. The system according to claim 23, wherein the insertion mechanism further comprises a track configured to guide the surgical clip during storage and during insertion of the surgical clip into the surgical apparatus.

25. The system according to claim 23, wherein the insertion mechanism further comprises a clip inserter configured to move the surgical clip from the insertion mechanism into the cavity of the surgical apparatus in a partially compressed state.
